(19) United States
(12) Patent Application Publication
(10) Pub. No.: US 2023/0091038 A1
(43) Pub. Date: Mar. 23, 2023

(54) SCALING TISSUE PRODUCTION THROUGH IMPROVED CONTROL OF MASS TRANSFER

(71) Applicant: Trustees of Tufts College, Medford, MA (US)

(72) Inventors: David L. Kaplan, Concord, MA (US); John Se Kit Yuen, Jr., Medford, MA (US)

(21) Appl. No.: 17/904,590

(22) PCT Filed: Feb. 18, 2021
(86) PCT No.: PCT/US2021/018629

§ 371 Date: Aug. 18, 2022

Related U.S. Application Data
(60) Provisional application No. 62/978,279, filed on Feb. 18, 2020.

(51) Int. Cl. C12N 5/071; C12M 3/00; C12M 1/00

(52) U.S. Cl. CPC C12N 5/0691; C12M 21/08; C12M 29/10; C12M 41/14; C12N 2521/00

(57) ABSTRACT

A method of forming a tissue. The method includes providing a source of a pre-tissue composition comprising endothelial cells. The method also includes perfusing a culture media into the pre-tissue composition using a plurality of primary channels and a plurality of secondary channels to form the tissue, wherein the endothelial cells are configured to form the secondary channels via vasculogenesis.

### CROSS-REFERENCE TO RELATED APPLICATIONS

The application is based on, claims priority to, and incorporates herein by reference in its entirety, U.S. Provisional Application Ser. No. 62/978,279, filed Feb. 18, 2020, and entitled “SCALING TISSUE PRODUCTION THROUGH IMPROVED CONTROL OF MASS TRANSFER.”

### STATEMENT REGARDING FEDERALLY SPONSORED RESEARCH

This invention was made with government support under grant DE-AR0001233 awarded by the Department of Energy. The government has certain rights in the invention.

### BACKGROUND

A major challenge in generating macroscale tissues has been the limitation in nutrient and O₂ diffusion to cells on the interior of in vitro generated constructs. The effective diffusion distance of nutrients and oxygen has been estimated to be only 100-200 microns, thus cells residing on the interior of large constructs cannot remain viable without a system in place for the internal delivery of nutrients/oxygen to sustain them. As a result, recreations of tissues in vitro that faithfully recapitulate native tissue organization have typically been limited to constructs of the micrometer and millimeter scale. However, larger constructs are highly desirable for many applications.

One prior strategy used to generate viable macroscale tissues incorporates channels that permit culture media flow into the tissue interior. This has been performed in skeletal muscle tissue engineering, where 10×10×1.9 mm tissues were perfused with hollow fibers (porous tubes). In another instance, the addition of bioprinted channels (ø 1 mm) supported fibroblasts and osteogenic mesenchymal stem cells in 1 cm thick tissues. However, the large size of these incorporated channels severely reduced tissue yields. For the hollow fiber (HF) system, close HF spacings and large HF diameters (480 μm) resulted in a muscle construct that was only 21% muscle by volume. Other attempts to improve upon this drawback have focused on the use of a smaller (ø120 μm) vascularized channels to perfuse adipose tissue, which could theoretically occupy only 1-1.5% of cultured tissue (assuming a 1 mm spacing between channels). Unfortunately, despite this improvement, such a system would be fairly complex, requiring an estimated 2500 channels to perfuse a 3D tissue of 5×5 cm cross section.

Another potential technique for perfusing macroscale tissues is to perform co-cultures with endothelial cells. In 3D culture, endothelial cells are able to form perfusable, capillary-like blood vessels that resemble in vivo microvasculature. This phenomenon has been leveraged to improve the performance and survival of various engineered muscle and adipose tissues post-implantation, and studies have reported successful anastomosis between host and engineered tissue vasculature. While endothelial incorporation has successfully vascularized engineered tissues, these studies have been limited to small constructs (millimeter scale), possibly because it takes too much time for vessel networks to form and deliver nutrients to interior cells when the volume of a construct is larger. Additionally, perfusing large constructs with small, capillary sized vessels may also result in mass transport complications, where the amount of media passing through a construct is not enough to sustain all its cells.

Thus, what is needed are novel systems and methods capable of perfusing large tissue engineered constructs while retaining high tissue yields and also reducing system complexity resulting from using a large number of channels.

### SUMMARY OF THE DISCLOSURE

The present disclosure addresses the aforementioned drawbacks by providing novel systems and methods for forming and perfusing large tissue engineered constructs using a unique perfusion arrangement that employs two distinct channel types.

In one aspect, the present disclosure provides a method of forming a tissue. The method may include providing a source of a pre-tissue composition comprising endothelial cells. The method may also include perfusing a culture media into the pre-tissue composition using a plurality of primary channels and a plurality of secondary channels to form the tissue, wherein the endothelial cells are configured to form the secondary channels via vasculogenesis.

In another aspect, the present disclosure provides a method of perfusing a tissue. The method may include perfusing a culture media into a tissue using a plurality of primary channels and a plurality of secondary channels, wherein the tissue comprises endothelial cells configured to form the secondary channels via vasculogenesis.

In one aspect, the present disclosure provides a method of perfusing a tissue. The method may include perfusing a culture media into a tissue using a plurality of arranged primary channels and a plurality of secondary channels, wherein culture media is provided to the tissue through the primary channels, and wherein one or more of the secondary channels spatially branch from the primary channels.

In another aspect, the present disclosure provides a system of forming a tissue. The system may include a source of a pre-tissue composition comprising endothelial cells configured to form the secondary channels via vasculogenesis. The system may also include a perfusion system configured to provide a culture media to the pre-tissue composition using a plurality of primary channels to form the tissue.

In yet another aspect, the present disclosure provides a system of perfusing a tissue. The system may include a perfusion system configured to provide a culture media to a tissue using a plurality of primary channels, wherein the tissue comprises endothelial cells configured to form a plurality of secondary channels via vasculogenesis.

These and other advantages and features of the present invention will become more apparent from the following detailed description of the preferred aspects of the present invention when viewed in conjunction with the accompanying drawings.

### BRIEF DESCRIPTION OF THE DRAWINGS

The drawings described herein are for illustrative purposes only of selected embodiments and not all possible implementations, and are not intended to limit the scope of any of the various embodiments. It is understood that the drawings are not drawn to scale.

FIG. 1 illustrates a process flowchart of a method of forming a tissue, in accordance with one aspect of the present disclosure.

FIG. 2 illustrates a process flowchart of a method of perfusing a tissue, in accordance with another aspect of the present disclosure.

FIG. 3 illustrates a process flowchart of a method of perfusing a tissue, in accordance with one aspect of the present disclosure.

FIG. 4A illustrates a depiction of a perspective view of a section of a tissue formed using the methods and systems described herein. FIG. 4B illustrates a depiction of a cross-sectional view of a section of a tissue formed using the methods and systems described herein.

FIG. 5 illustrates a depiction of a system for forming or perfusing a tissue, in accordance with multiple aspects of the present disclosure.

FIG. 6 illustrates a schematic cross-sectional view of a section of a tissue formed using the methods and systems described herein.

FIG. 7A depicts Brightfield image results obtained using Co-Culture Media Formulation 2 in the experiment of Example 1. FIG. 7B depicts Brightfield image results obtained using Co-Culture Media Formulation 1 in the experiment of Example 1. FIG. 7C depicts Brightfield image results obtained using Co-Culture Media Formulation 1 including 10 nM of dexamethasone and 30 ng/ml of IGF-1 in the experiment of Example 1.

FIG. 8A depicts BODIPY lipid staining results obtained using Co-Culture Media Formulation 1 including 10 nM of dexamethasone and 30 ng/ml of IGF-1 in the experiment of Example 1. FIG. 8B illustrates depicts BODIPY lipid staining results obtained using Co-Culture Media Formulation 2 including 10 nM of dexamethasone and 30 ng/ml of IGF-1 in the experiment of Example 1. FIG. 8C illustrates a chart quantifying the BODIPY lipid staining results in the experiment of Example 1.

FIG. 9A depicts CD31 staining of GFP-HUVECs obtained using Co-Culture Media Formulation 1 with Dexamethasone and IGF-1 in the experiment of Example 1. FIG. 9B depicts CD31 staining of GFP-HUVECs obtained using Co-Culture Media Formulation 1 with Dexamethasone, IGF-1, and 100 ug/ml Intralipid in the experiment of Example 1. FIG. 9C illustrates a chart quantifying the CD31 staining results of the experiment of Example 1.

FIG. 10A depicts Brightfield image results obtained for an adipose-endothelial 3D Co-Culture in the experiment of Example 1. FIG. 10B depicts CD31 staining results obtained for an adipose-endothelial 3D Co-Culture in the experiment of Example 1.

FIG. 11A depict Myosin heavy chain (MHC) staining results of C2C12 mouse myoblasts obtained using a conventional horse serum-based differentiation medium in the experiment of Example 2. FIG. 11B depict Myosin heavy chain (MHC) staining results of C2C12 mouse myoblasts obtained using the developed Muscle-Endothelial Co-Culture Media formulation in the experiment of Example 2. FIG. 11C illustrates a chart quantifying the degree of myotube formation measured in the staining results of FIGS. 11A-11B.

FIG. 12A depicts myosin heavy chain (MHC) and DAPI nuclear staining for a 3D muscle culture with a horse serum-based differentiation media in the experiment of Example 2. FIG. 12B depicts myosin heavy chain (MHC) and DAPI nuclear staining for a 3D muscle culture with the developed Muscle-Endothelial Co-Culture Media formulation in the experiment of Example 2. FIG. 12C depicts phalloidin staining results for a 3D muscle culture with a horse serum-based differentiation media in the experiment of Example 2. FIG. 12D depicts phalloidin staining results for a 3D muscle culture with the developed Muscle-Endothelial Co-Culture Media formulation in the experiment of Example 2.

FIG. 13 illustrates a schematic of an experimental bioreactor loop perfusion system used in the experiment of Example 3.

FIG. 14A depicts live/dead staining of endothelial cells cultured in a hydrogel perfused by a single hollow fiber channel system. Cell growth can be seen tracking the outflow of the culture media. FIG. 14B depicts live/dead staining of endothelial cells cultured in a hydrogel perfused by a single hollow fiber channel system. Network or proto-network formation can be ob served.

### DETAILED DESCRIPTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

Specific structures, devices and methods relating to macroscale tissue formation and perfusion are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, the terms “perfuse” and “perfusing” have their ordinary meaning in the art. For instance, “perfusing a tissue” may refer to pumping of a fluid through the tissue. Perfusion may be measured as the rate at which the fluid is delivered to tissue, or by the volume of fluid per unit time per unit tissue mass.

As used herein, the term “vasculogenesis” has its ordinary meaning in the art. For instance, “vasculogenesis” can refer to the formation of new channels or blood vessels, when there are no pre-existing ones in the area.

As used herein, the term “biocompatible” refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo.

Prior systems tissue perfusion strategies have been shown to exhibit considerable drawbacks. Macroscale tissues perfused with only channels either lose too much tissue volume or require a very high number of smaller channels, while in vitro endothelial vessels are capillary sized (ø30 μm) and can self-assemble in culture, but lack the transport capabilities to support macroscale tissue growth.

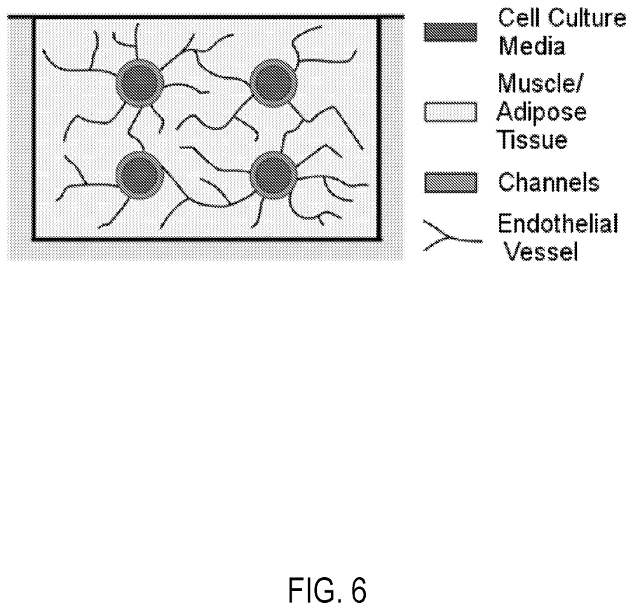

Described herein are systems and methods related to the production of macroscale tissues by incorporating a dual perfusion strategy where nutrients and O₂ are delivered to large tissues through both microchannels and capillary-sized endothelial vessels (FIG. 6E). The addition of endothelial cells to channel-perfused tissues allows for wider channel spacings, increasing tissue yield and reducing bioreactor complexity by reducing the number of channels required for perfusion. Cells too distant from the more disperse arrangement are linked to the channels by capillary-like vessels, as a result of vasculogenesis from the co-cultured endothelial cells. At the same time, channel incorporation means that there will already be partial perfusion in the 3D tissue at the beginning of culture, and cells only need to wait for endothelial cells to vascularize the space in between the channels, rather than the entire construct. Channels also ensure that enough culture media perfuses large scale tissue constructs, overcoming the mass transport issue in a tissue that would otherwise be perfused solely by capillary-sized vessels. Lastly, the transition from larger channels to small capillary vessels is more biomimetic, much like the hierarchy of size seen in the human circulatory system with arteries and capillaries.

In this manner, the systems and methods herein alleviate the drawbacks associated with systems that use only either channels or endothelial vessels. While endothelial networks can take days to form, primary channels provide perfusion during the beginning of culture. Additionally, endothelial cell capillaries suffer from potential mass transport problems, and the larger diameters of channels permit more media flow.

The dual perfusion strategy of the present disclosure stands to significantly improve upon current large tissue engineering techniques, enabling in vitro generated tissues to be generated at scales large enough to be applicable in numerous fields/industries. For example, the production of macroscale muscle tissue could be used as grafts in cases of treat volumetric muscle loss, where muscle damage is severe enough that its self-regenerating components are also destroyed. The generation of macroscale adipose tissue allows for the repair of soft tissue defects in patients, or to add bulk to desired regions of the body during cosmetic/plastic surgery. The emerging field of in vitro meat (where tissue is generated in vitro to produce meat for eating without animal slaughter) also stands to benefit from the present disclosure, as the field is based on the ability to produce muscle and adipose tissues at scale. One of skill in the art would readily recognize further applications in addition to these exemplary uses.

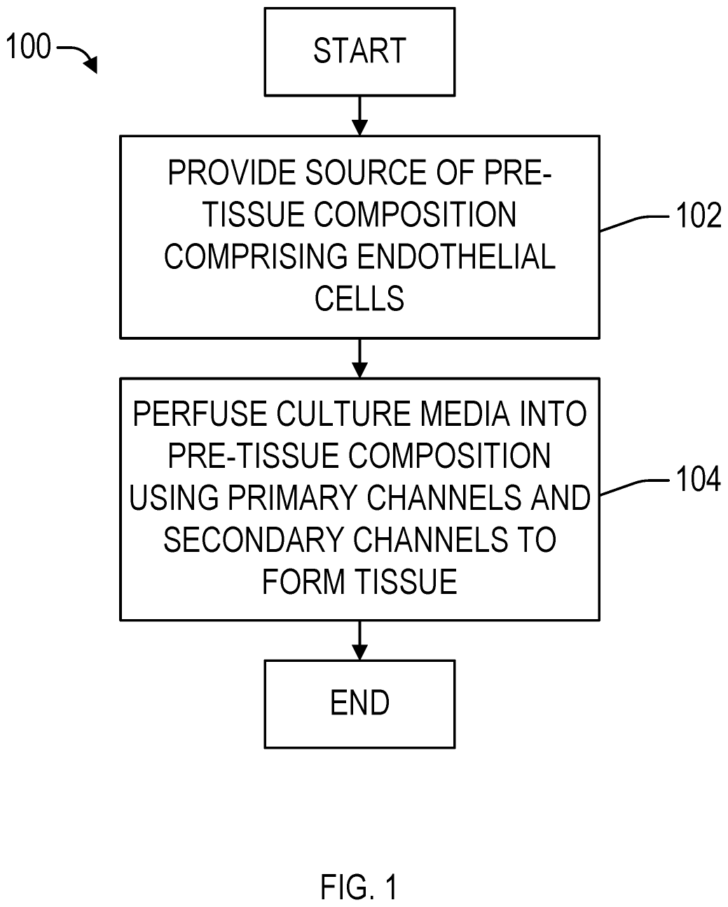

FIG. 1 depicts a method 100 of forming a tissue. The method includes a first step 102 of providing a source of a pre-tissue composition comprising endothelial cells; and a second step 104 of perfusing a culture media into the pre-tissue composition using a plurality of primary channels and a plurality of secondary channels to form the tissue, wherein the endothelial cells are configured to form the secondary channels via vasculogenesis.

The primary channels of the method 100 may have an average outer diameter of less than 800, 500, 400, 300, 200, 150, 100, or 50 μm. The primary channels may be arranged to extend through the pre-tissue composition in a substantially parallel configuration. The primary channels may be arranged in a pattern suitable to provide adequate perfusion of the culture media to all areas of the pre-tissue composition. For instance, the channels may be arranged in a grid-like pattern, having roughly equidistant spacing between each other. The average cross-sectional channel density of the tissue may be defined as the number of primary channels per area of a cross section taken perpendicular to the primary channels. The average cross-sectional channel density of the tissue may be less than 100, 50, 30, 20, 10, 8, or 4 primary channels per square centimeter. The volume of the plurality of primary channels may comprises less than 20%, 10%, 5%, 3%, 2%, 1%, or 0.5% of the total volume of the product tissue.

The primary channels of the method 100 may be formed of a biocompatible or biodegradable material. For instance, the primary channels may be formed of a biocompatible polymer. The primary channels may specifically comprise regenerated silk fibroin. The channels may be specifically preformed prior to the initiation of the method, and the method step of perfusing the culture media may initially occur solely through the primary channels prior to the formation of the secondary channels. The channels may be hollow fiber membranes which extend through the pre-tissue composition. The primary channels may be porous to permit perfusion of the culture media. Alternatively, the channels may be non-porous and rely on diffusion to perfuse the tissue.

The secondary channels of the method 100 may be smaller than the primary channels and may have an average outer diameter of less than 70, 50, 40, or 30 μm. The secondary channels may specifically have an average outer diameter between 5 μm and 70 μm, between 10 μm and 50 μm, between 20 μm and 40 μm, or about 30 μm. One or more of the secondary channels may extend from one or more of the primary channels into the interchannel space of the pre-tissue composition, where interchannel refers to the tissue space between primary channels. In this manner, the secondary channels may serve to expand the range of perfusion of the primary channels, allowing for increased spacing between these larger channels. In some cases, one or more of the secondary channels may fluidly connect one or more of the primary channels.

The secondary channels may be formed by endothelial cells ability to initiate vasculogenesis. For instance, endothelial precursor cells may migrate and differentiate in response to local cues, such as growth factors and extracellular matrices within the tissue to form new channels, which resemble capillaries. These vascular trees may then be extended through angiogenesis in the tissue. The pre-tissue composition may comprise a specific amount of endothelial cells to allow for sufficient perfusion of culture media. Additional biomolecules such as growth factors may be included in the pre-tissue composition or later added to support the formation of the secondary channels.

The culture media may be formulated with nutrients and O₂ to support cell growth in the tissue. The culture media may have about the same initial oxygen concentration as typical arterial blood of the genetic source of the tissue. The culture media may be continuously perfused to the pre-tissue composition over a set period of time or until a specific tissue volume is reached. Alternatively, the culture media may be provided in batch pulses either consistently or as needed. The cells of the tissue may be monitored and the perfusion rate adjusted accordingly. Similarly, the composition of the culture media may be adjusted over time to account for cell health concerns. For instance, a growth factor concentration in the culture media may be increased as needed.

The tissue formed by the method may be dependent on the initial cell type and amount present in the pre-tissue composition. The product tissue may be formed of mammalian cells. The tissue may be human tissue for use in various application such as for a skin graft or organ transplant. The tissue may be predominantly muscle tissue. The tissue may be formed of non-human cells and be specifically crafted for consumption as a food product. One of skill in the art will recognize numerous additional applications suitable for the present tissue formation techniques.

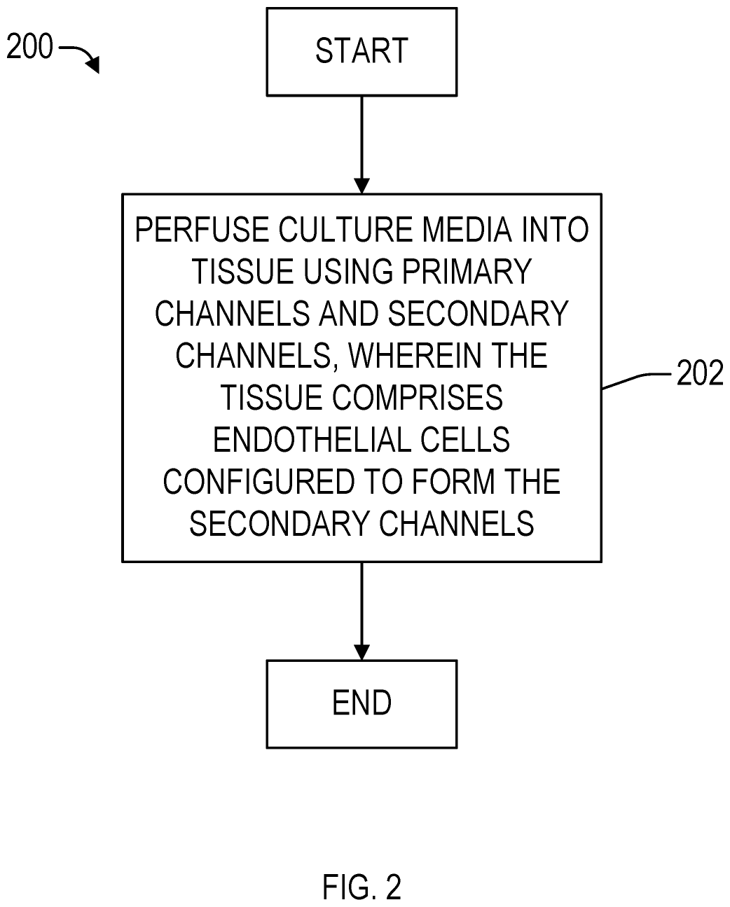

FIG. 2 depicts a method 200 of perfusing a tissue. The method includes a first step 202 of perfusing a culture media into a tissue using a plurality of primary channels and a plurality of secondary channels, wherein the tissue comprises endothelial cells configured to form the secondary channels via vasculogenesis. The method of perfusion 200 may utilize any compositions, configurations, and/or method steps of any of the elements of methods or systems described herein.

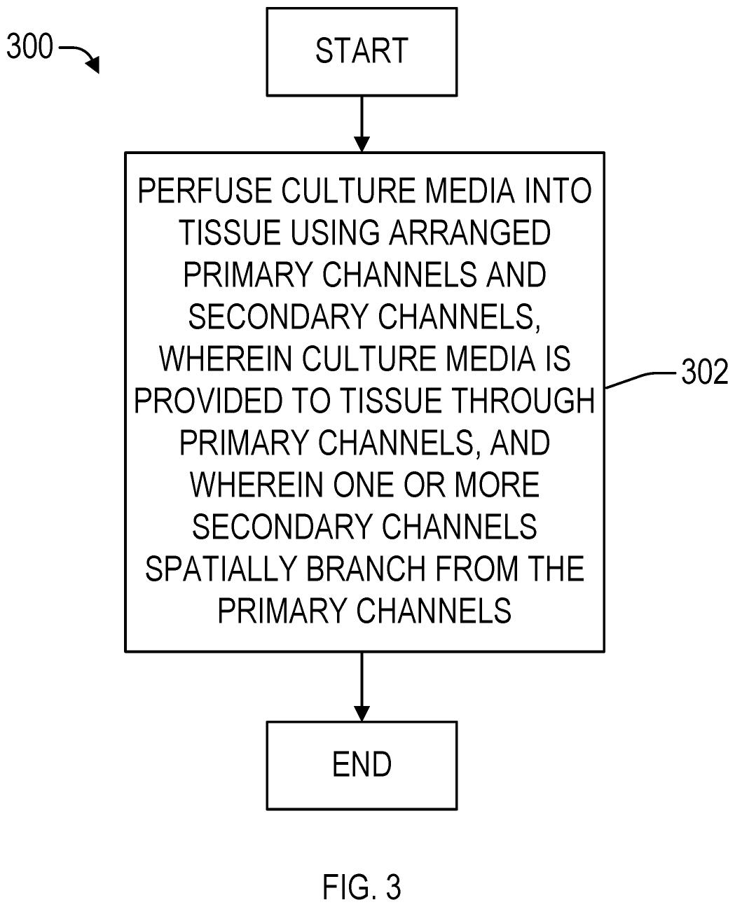

FIG. 3 depicts another method 300 of perfusing a tissue. The method includes first step 302 of perfusing a culture media into a tissue using a plurality of arranged primary channels and a plurality of secondary channels, wherein culture media is provided to the tissue through the primary channels, and wherein one or more of the secondary channels spatially branch from the primary channels. The method of perfusion 300 may utilize any compositions, configurations, and/or method steps of any of the elements of methods or systems described herein.

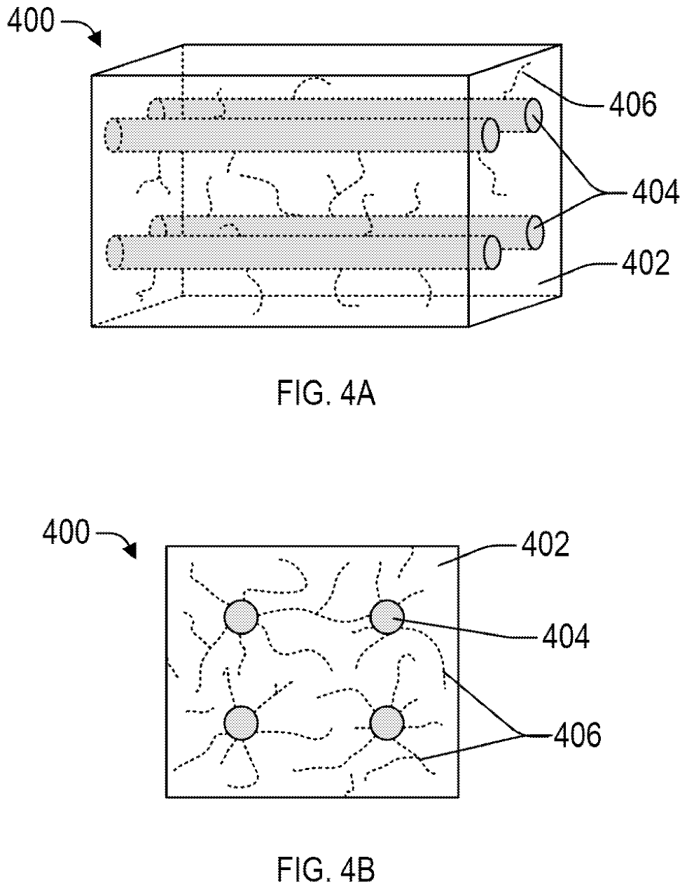

FIGS. 4A and 4B depict separate views of a section of a tissue 400 formed using methods and/or systems described herein. The section 400 contains several primary channels 404 which extend through the interchannel tissue 402. Also depicted are numerous secondary channels 406 within the tissue 402. The secondary channels 406 can be seen to branch off of the primary channels 404, connect the primary channels 404, exist as standalone channels within the tissue 402, or connect the exterior of the section to the interchannel tissue 402.

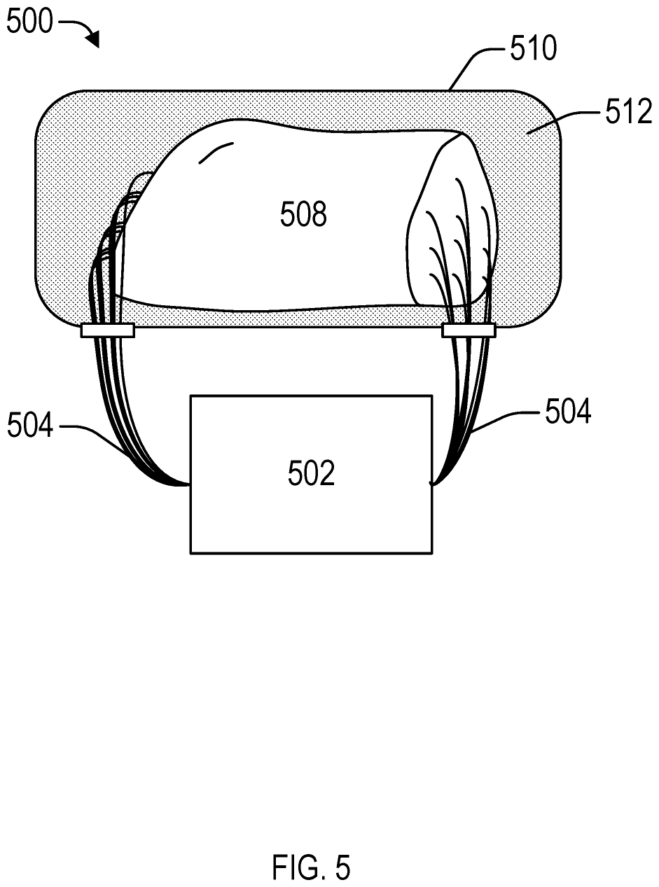

FIG. 5 depicts a system 500 configured to either form or perfuse a tissue 508. In one aspect, the system 500 is configured to form a tissue 508. The system 500 includes a source of a pre-tissue composition (not depicted, as the tissue has already been formed) comprising endothelial cells configured to form the secondary channels via vasculogenesis. The system also includes a perfusion system 502 configured to provide a culture media to the pre-tissue composition using a plurality of primary channels 504 to form the tissue 508.

In another aspect, the system 500 is configured to perfuse a tissue 508. In this aspect, the system 500 includes a perfusion system 502 configured to provide a culture media to a tissue 508 using a plurality of primary channels 504, wherein the tissue 508 comprises endothelial cells configured to form a plurality of secondary channels via vasculogenesis.

In both aspects, the system 500 may comprise an incubator 510 configured to house the tissue at conditions suitable for growth. The incubator may comprise media 512 suitable for cell growth or maintenance. The incubator 510 may be configured to maintain a substantially parallel arrangement of the primary channels 504. In both aspects, the system may comprise a source of culture media and/or a pump configured to provide the culture media to the tissue using the primary channels 504. Such a pump may be configured to continuously provide the culture media to the pre-tissue composition. The primary channels 504 may be arranged to extend through the tissue in a substantially parallel configuration. The primary channels 504 may be tubular constructs. The average cross-sectional channel density of the tissue 508 may be less than 20 primary channels per square centimeter, or less than 10 primary channels per square centimeter. The pre-tissue composition or the tissue 508 may comprise human tissue cells. Alternatively, the pre-tissue composition or the tissue 508 may comprise bovine, porcine, or poultry tissue cells.

In both aspects, the system 500 may utilize any compositions or configurations of any of the elements of methods described herein.

### EXAMPLES

The following Examples are provided in order to demonstrate and further illustrate certain embodiments and aspects of the present disclosure and are not to be construed as limiting the scope of the disclosure.

Proof-of-concept experiments were performed in order demonstrate the efficacy of the systems and methods described herein. In these experiments, specific techniques for co-culturing adipose and endothelial, as well as muscle and endothelial cells were established. The results of these experiments provide fundamental knowledge that is useful for effectively culturing endothelial cells with myocytes or adipocytes in perfused 3D tissue cultures.

### Example 1

Experiments were conducted to establish suitable culture conditions and co-culture media formulations for adipose and endothelial cells. Multiple pre-tissue compositions, also referred to as cell culture scaffold formulations, and co-culture media formulations were developed.

Cell Culture Scaffold Formulation 1 included 0.5 mg/ml GrowDex (nanofibrillar cellulose hydrogel), 1 mg/ml Matrigel, 1 mg/ml Fibrin, and 1.25 U/ml thrombin. Cell Culture Scaffold Formulation 2 included 1.25 mg/ml Matrigel, 1.25 mg/ml Fibrin, and 1.25 U/ml thrombin. Co-Culture Media Formulation 1 included endothelial Cell Growth Medium MV 2 (Promocell), with an additional 9.5 ng/ml VEGF165, 1× of Animal-Free Insulin-Transferrin-Selenium-Ethanolamine (Invitria), 0.5 mg/ml AlbuMAX I (Gibco), 100 ug/ml Intralipid OR 160 uM oleic acid-methyl-β-cyclodextrin complex, 100 U/ml Penicillin, 100 ug/ml streptomycin. Optionally, 10 nM-1 uM Dexamethasone and 30 ng/ml IGF-1 could be included in Co-Culture Media Formulation 1. The Endothelial Cell Growth MV 2 used in Co-Culture Media Formulation 1 contains EBM, 5% FBS, 5 ng/ml EGF, 10 ng/ml FGFb, 20 ng/ml IGF-1, 0.5 ng/ml VEGF165, 1 ug/ml ascorbic acid, 0.2 ug/ml hydrocortisone, wherein EBM is an Endothelial Basal Medium from Promocell. Optionally, Co-Culture Media Formulation 1 can substitute MCDB 131 for EBM. The EBM was expected to provide about 30 nM d-Biotin, 17 μM L-Ascorbic Acid Phosphate, 0.03 Selenite, and 5.55 mM Glucose, and to contain no BSA, oleic acid, insulin, or transferrin. Co-Culture Media Formulation 2 included EBM with 10% FBS, 10 ug/ml insulin, 100 ug/ml Intralipid or 160 uM oleic acid-methyl-β-cyclodextrin complex, 100 U/ml Penicillin, and 100 ug/ml streptomycin, wherein Intralipid is a sterile lipid emulsion derived from soybean oil.

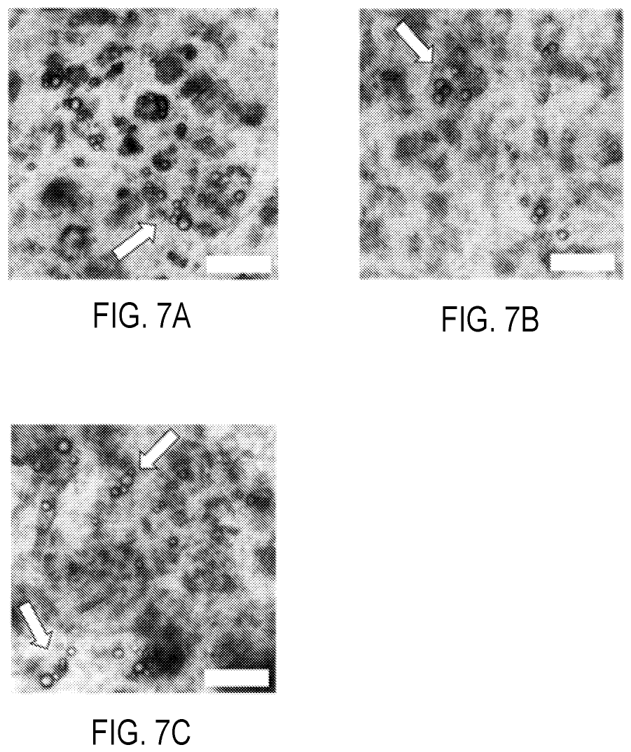

FIGS. 7A-7C depict Brightfield images of 3T3-L1 adipogenic cells cultured in Co-Culture Media Formulations 1 and 2. The experiment used 5 million 3T3-L1s and the images were captured after 7 days for MFG gel. FIG. 7A depicts the results of Formulation 2. FIG. 7C depicts the results of Formulation 1, which included 10 nM of dexamethasone and 30 ng/ml of IGF-1. FIG. 7B depicts the results of Formulation 1 without the addition of dexamethasone, IGF-1, and oleic acid-methyl-β-cyclodextrin complex/intralipid. The depicted scale bars are 50 micrometers.

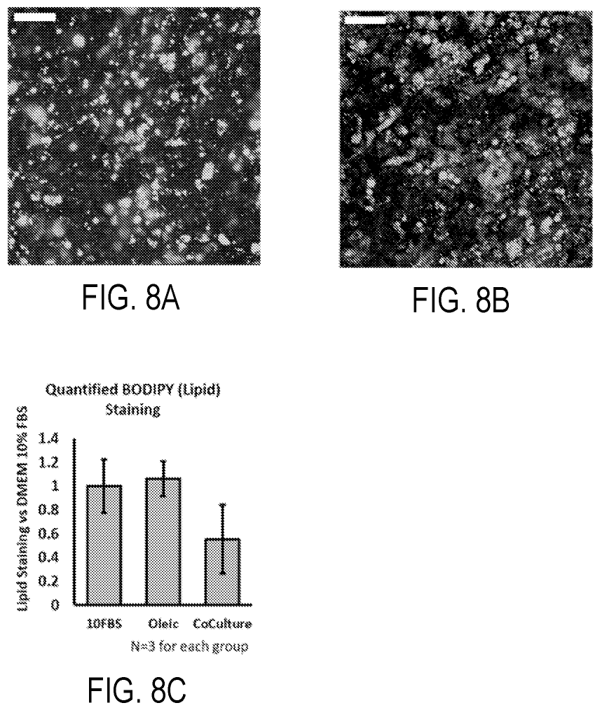

FIGS. 8A-8C depict BODIPY lipid staining with DAPI of the same experiment of FIGS. 7A-7C involving the 3T3-L1 adipogenic cells cultured in in Co-Culture Media Formulations 1 and 2. The experiment used 5 million 3T3-L1s and BODIPY lipid staining occurred after 21 days for MFG gel. FIG. 8A depicts the results of Formulation 1. FIG. 8B depicts the results of Formulation 2. The depicted scale bars are 100 micrometers. FIG. 8C depicts the quantified BODIPY lipid staining results, wherein the “CoCulture” bar refers to Formulation 1 without the addition of dexamethasone, IGF-1 and oleic acid-methyl-β-cyclodextrin complex/intralipid.

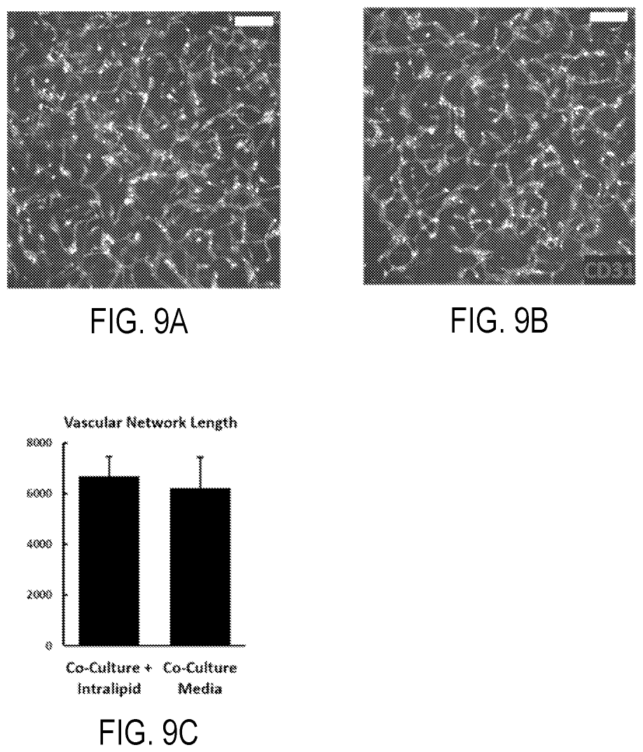

FIGS. 9A-9C depict CD31 staining of GFP-HUVECs (human umbilical vein endothelial cells) that demonstrate vascular network formation. The experiment used 2 million GFP-HUVECs, 400,000 10T1/2s per ml and staining occurred after 7 days for MFG gel. FIG. 9A depicts the results using Formulation 1 with Dexamethasone and IGF-1. FIG. 9B depicts the results using Formulation 1 with Dexamethasone, IGF-1, and 100 ug/ml Intralipid. The depicted scale bars are 200 micrometers. FIG. 9C depicts the quantified results of the experiment, wherein the network length is given in arbitrary units. These results confirm that lipid addition to the co-culture media formulation did not affect vessel formation.

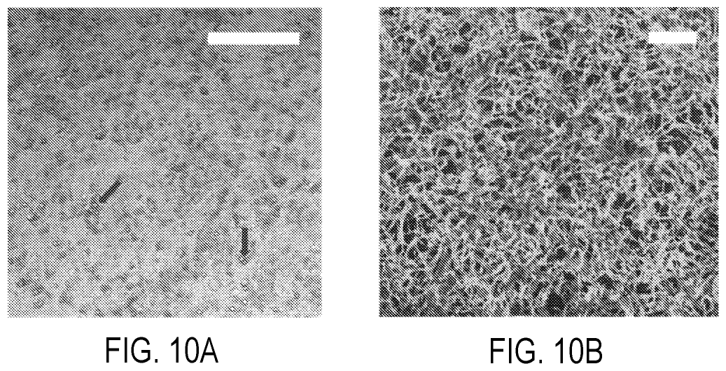

FIGS. 10A-10B depict Bright field images and CD31 staining for an adipose-endothelial 3D Co-Culture. The experiment used 5 million 3T3-L1s, 3 million HUVECs, and 600,000 10T1/2s per ml and the images and staining occurred after 7 days for Matrigel-Fibrin gel. Adipose-Endothelial 3D Co-Culture. FIG. 10A depicts a Brightfield image of numerous lipid droplets, with examples highlighted by arrows. FIG. 10B depicts CD31 staining of the endothelial vascular network formation. The depicted scale bars are 200 micrometers.

### EXAMPLE 2

Experiments were conducted to establish suitable culture conditions and co-culture media formulations for muscle and endothelial cells. A pre-tissue composition, also referred to as scaffold formulations, and a co-culture media formulation were developed.

The Muscle-Endothelial Scaffold Formulation included 1.25 mg/ml Matrigel, 1.25 mg/ml Fibrin, and 1.25 U/ml thrombin. The Muscle-Endothelial Co-Culture Media Formulation included Endothelial Cell Growth Medium MV 2 (Promocell), with an additional 9.5 ng/ml VEGF165, 1× of Animal-Free Insulin-Transferrin-Selenium-Ethanolamine (Invitria), 0.5 mg/ml AlbuMAX I (Gibco), 30 ng/ml IGF-1, 10 nM Dexamethasone, and 100 U/m Penicillin, 100 ug/ml streptomycin. Optionally, the Muscle-Endothelial Co-Culture Media Formulation can further include 250 nM -1 uM Sphingosine-1-Phosphate and 10-200 ng/ml Prostaglandin E2 or 100 nM SW033291.

The results of the experiment demonstrate that, through only an optimization of the culture media, considerably improved myogenesis in terms of degree of myotube formation (fusion index) and the size of 3D cultured constructs was achieved.

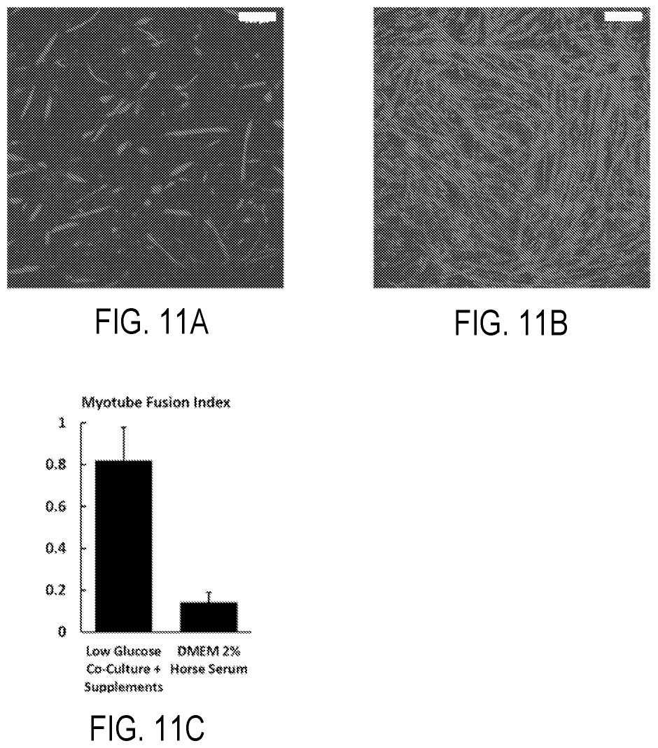

FIGS. 11A-11C depict experimental results for Myosin heavy chain (MHC) staining of C2C12 mouse myoblasts. The experiment used 40,000 cells/cm² and the staining occurred after 7 days for a 2D culture. FIG. 11A depicts the results for a conventional horse serum-based differentiation medium. FIG. 11B depicts the results for the Muscle-Endothelial Co-Culture Media formulation with 250 nM sphingosine-1-phosphate. The depicted scale bars are 200 micrometers. FIG. 11C quantifies the degree of myotube formation measured in FIGS. 11A-11B, wherein the Muscle-Endothelial Co-Culture Media formulation is labeled as “Low Glucose Co-Culture+Supplements”.

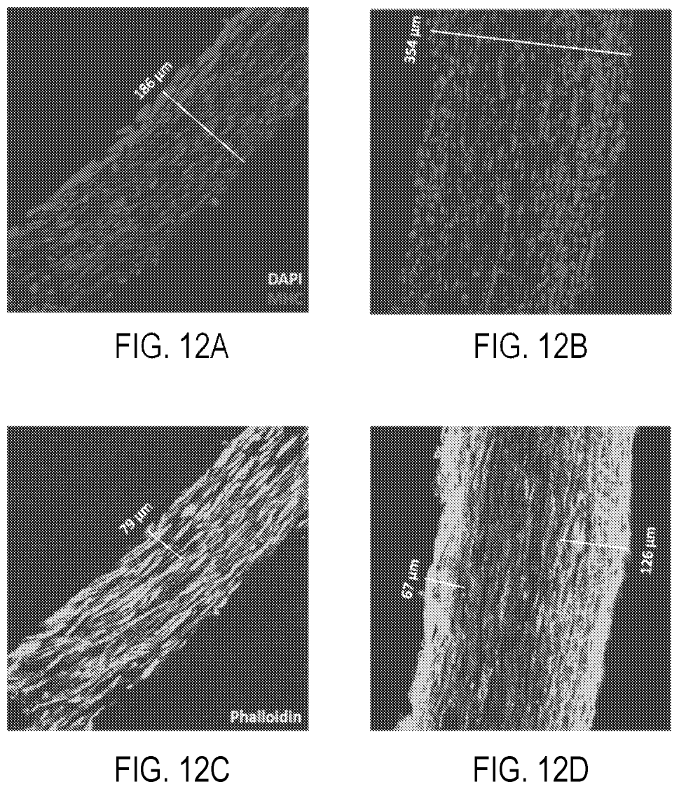

FIGS. 12A-12D depict staining results for a comparison of traditional horse serum-based differentiation media to the Muscle-Endothelial Co-Culture Media formulation. The experiment used C2C12 mouse myoblasts at a concentration of 10 million/ml and the staining occurred after 14 days for a 3D culture of 1.25 mg/ml Matrigel and 1.25 mg/ml fibrin. FIG. 12A depicts myosin heavy chain (MHC) and DAPI nuclear staining for a 3D muscle culture with a horse serum-based differentiation media. FIG. 12B depicts myosin heavy chain (MHC) and DAPI nuclear staining for a 3D muscle culture with the developed Muscle-Endothelial Co-Culture Media formulation with 250 nM sphingosine-1-phosphate. FIGS. 12C-12D depict the same views as FIGS. 12A-12B, except with visualization of the actin cytoskeleton via phalloidin, wherein FIG. 12C is the horse serum-based media and FIG. 12D is the developed Muscle-Endothelial Co-Culture Media formulation. It can be seen in FIG. 12D that, once a muscle 3D construct increases in size, cells on the interior experience diminished growth (implied by decreased cytoskeleton presence) due to nutrient and oxygen transfer limitations.

### EXAMPLE 3

In order to further test the techniques described herein, an experimental, single-channeled bioreactor system was developed in order to observe vascular network formation around a channel incorporated into the 3D co-culture hydrogels.

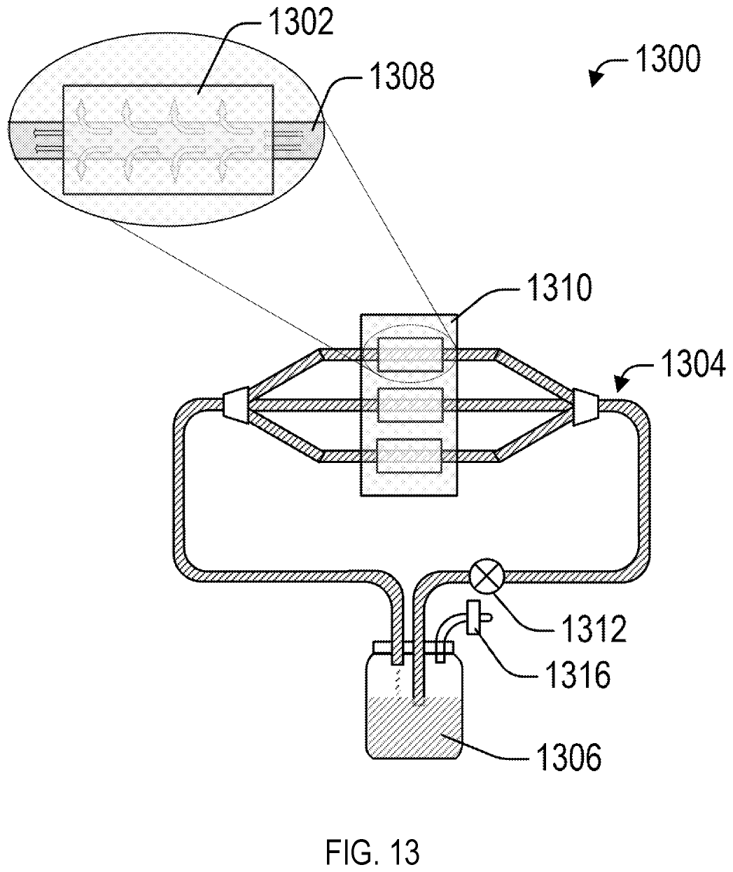

FIG. 13 depicts a schematic of the experimental bioreactor loop perfusion system 1300 that was developed to investigate vascular network formation around single channels. The experimental system 1300 included a source 1302 of a pre-tissue composition comprising endothelial cells, specifically a hydrogel matrix with endothelial cells. A perfusion system 1304 provided culture media from a culture media source 1306 to the pre-tissue composition 1302 using individual channels 1308 in a hollow fiber chamber 1310. A magnified callout view of the individual channels 1308 show inlet culture media perfusing into the hydrogel and outlet culture media being removed. A pump 1312, which was either a peristaltic pump with a high flow rate or a syringe pump with a low flow rate, was used to ensure constant circulation of the culture media through the perfusion system 1304. A gas exchanger 1316 was used to maintain the suitability of the culture media during the experiment.

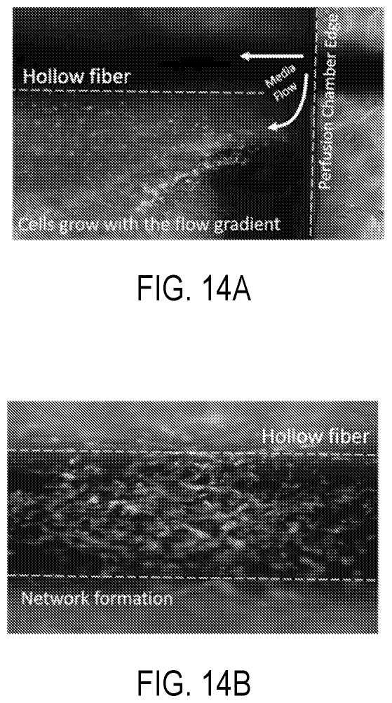

FIGS. 14A-14B depict live/dead staining of endothelial cells cultured in the hydrogel perfused by a single hollow fiber channel of the system of FIG. 13. Specifically, human dermal microvascular endothelial cells (HMEC-1s) at a concentration of 3 million cells/mL and rat smooth muscle cells (A10s) at a concentration of 0.6 million cells/mL were perfused at a flowrate of 4 μL/min for a hydrogel formed of 1 mg/mL fibrin, 1 mg/mL Matrigel, and 0.5 mg/mL GrowDex. As can be seen in FIG. 14A, cell growth can be seen tracking the outflow of the culture media from the hollow fiber. Additionally, as shown in FIG. 14B, network or proto-network formation can be observed around the hollow fiber.

The present disclosure has described one or more preferred embodiments, and it should be appreciated that many equivalents, alternatives, variations, and modifications, aside from those expressly stated, are possible and within the scope of the invention.

We claim:

1. A method of forming a tissue, the method comprising:
providing a source of a pre-tissue composition comprising endothelial cells; and
perfusing a culture media into the pre-tissue composition using a plurality of primary channels and a plurality of secondary channels to form the tissue, wherein the endothelial cells are configured to form the secondary channels via vasculogenesis.

2. The method of claim 1, wherein the primary channels have an average outer diameter of less than 400 μm.

3. The method of claim 1 or 2, wherein the primary channels have an average outer diameter of less than 150 μm.

4. The method of any one of the preceding claims, wherein the primary channels are arranged to extend through the pre-tissue composition in a substantially parallel configuration.

5. The method of any one of the preceding claims, wherein one or more of the secondary channels extends from one or more of the primary channels into the interchannel space of the pre-tissue composition.

6. The method of any one of the preceding claims, wherein one or more of the secondary channels fluidly connects one or more of the primary channels.

7. The method of any one of the preceding claims, wherein the average cross-sectional channel density is less than 20 primary channels per square centimeter.

8. The method of any one of the preceding claims, wherein the average cross-sectional channel density is less than 10 primary channels per square centimeter.

9. The method of any one of the preceding claims, wherein the primary channels are porous.

10. The method of any one of the preceding claims, wherein the secondary channels have an average outer diameter between 10 μm and 50 μm.

11. The method of any one of the preceding claims, wherein the method step of perfusing the culture media initially occurs solely through the primary channels prior to the formation of the secondary channels.

12. The method of any one of the preceding claims, wherein culture media is continuously perfused to the pre-tissue composition.

13. The method of any one of the preceding claims, wherein the tissue is predominantly muscle tissue.

14. The method of any one of the preceding claims, wherein the volume of the plurality of primary channels comprises less than 5% of the total volume of the product tissue.

15. The method of any one of the preceding claims, wherein the volume of the plurality of primary channels comprises less than 1% of the total volume of the product tissue.

16. A method of perfusing a tissue, the method comprising:
perfusing a culture media into a tissue using a plurality of primary channels and a plurality of secondary channels, wherein the tissue comprises endothelial cells configured to form the secondary channels via vasculogenesis.

17. The method of claim 16, wherein the primary channels have an average outer diameter of less than 400 μm.

18. The method of claim 17, wherein the primary channels have an average outer diameter of less than 150 μm.

19. The method of any one of the preceding claims, wherein the primary channels are arranged to extend through the tissue in a substantially parallel configuration.

20. The method of any one of the preceding claims, wherein one or more of the secondary channels extends from one or more of the primary channels into the interchannel space of the tissue.

21. The method of any one of the preceding claims, wherein one or more of the secondary channels fluidly connects one or more of the primary channels.

22. The method of any one of the preceding claims, wherein the average cross-sectional channel density is less than 20 primary channels per square centimeter.

23. The method of any one of the preceding claims, wherein the average cross-sectional channel density is less than 10 primary channels per square centimeter.

24. The method of any one of the preceding claims, wherein the primary channels are porous.

25. The method of any one of the preceding claims, wherein the secondary channels have an average outer diameter between 10 μm and 50 μm.

26. The method of any one of the preceding claims, wherein the culture media has about the same initial oxygen concentration as typical arterial blood of the genetic source of the tissue.

27. The method of any one of the preceding claims, wherein culture media is continuously perfused to the tissue.

28. The method of any one of the preceding claims, wherein the tissue is predominantly muscle tissue.

29. A method of perfusing a tissue, the method comprising:
perfusing a culture media into a tissue using a plurality of arranged primary channels and a plurality of secondary channels, wherein culture media is provided to the tissue through the primary channels, and wherein one or more of the secondary channels spatially branch from the primary channels.

30. The method of claim 29, wherein the secondary channels have a smaller average diameter than the primary channels.

31. The method of claim 30, wherein the primary channels have an average outer diameter between 100 μm and 400 μm.

32. The method of any one of the preceding claims, wherein the secondary channels have an average outer diameter between 10 μm and 50 μm.

33. The method of any one of the preceding claims, wherein the primary channels are arranged to extend through the tissue in a substantially parallel configuration.

34. The method of any one of the preceding claims, wherein one or more of the secondary channels fluidly connects one or more of the primary channels.

35. The method of any one of the preceding claims, wherein the average cross-sectional channel density is less than 20 primary channels per square centimeter.

36. The method of any one of the preceding claims, wherein the average cross-sectional channel density is less than 10 primary channels per square centimeter.

37. The method of any one of the preceding claims, wherein the primary channels are porous.

38. The method of any one of the preceding claims, wherein the culture media has about the same initial oxygen concentration as the arterial blood of the genetic source of the tissue.

39. The method of any one of the preceding claims, wherein culture media is continuously perfused to the tissue.

40. The method of any one of the preceding claims, wherein the tissue is predominantly muscle tissue.

41. A tissue formed using the method of any one of the preceding claims.

42. A system of forming a tissue, the system comprising:
a source of a pre-tissue composition comprising endothelial cells configured to form the secondary channels via vasculogenesis; and
a perfusion system configured to provide a culture media to the pre-tissue composition using a plurality of primary channels to form the tissue.

43. The system of claim 42 further comprising:
an incubator configured to house the tissue at conditions suitable for growth.

44. The system of claim 43, wherein the incubator is configured to maintain a substantially parallel arrangement of the primary channels.

45. The system of any one of claims 42 to the immediately preceding claim, wherein the perfusion system comprises:
a source of culture media; and
a pump configured to provide the culture media to the tissue using the plurality of primary channels.

46. The system of any one of claims 42 to the immediately preceding claim, wherein a pump is configured to continuously provide the culture media to the pre-tissue composition.

47. The system of any one of claims 42 to the immediately preceding claim, wherein the primary channels are arranged to extend through the tissue in a substantially parallel configuration.

48. The system of any one of claims 42 to the immediately preceding claim, wherein the primary channels are tubular constructs.

49. The system of any one of claims 42 to the immediately preceding claim, wherein the average cross-sectional channel density is less than 20 primary channels per square centimeter.

50. The system of any one of claims 42 to the immediately preceding claim, wherein the average cross-sectional channel density is less than 10 primary channels per square centimeter.

51. The system of any one of claims 42 to the immediately preceding claim, wherein the pre-tissue composition comprises human tissue cells.

52. The system of any one of claims 42 to the immediately preceding claim, wherein the pre-tissue composition comprises bovine, porcine, or poultry tissue cells.

53. A system of perfusing a tissue, the system comprising:
a perfusion system configured to provide a culture media to a tissue using a plurality of primary channels, wherein the tissue comprises endothelial cells configured to form a plurality of secondary channels via vasculogenesis.

54. The system of claim 53 further comprising an incubator configured to house the tissue at conditions suitable for cell maintenance.

55. The system of claim 54, wherein the incubator is configured to maintain a substantially parallel arrangement of the primary channels.

56. The system of any one of claims 53 to the immediately preceding claim, wherein the perfusion system comprises:
a source of culture media; and
a pump configured to provide the culture media to the tissue using the plurality of primary channels.

57. The system of any one of claims 53 to the immediately preceding claim, wherein the primary channels are arranged to extend through the tissue in a substantially parallel configuration.

58. The system of any one of claims 53 to the immediately preceding claim, wherein the primary channels are tubular constructs.

59. The system of any one of claims 53 to the immediately preceding claim, wherein the average cross-sectional channel density is less than 20 primary channels per square centimeter.

60. The system of any one of claims 53 to the immediately preceding claim, wherein the average cross-sectional channel density is less than 10 primary channels per square centimeter.

61. The system of any one of claims 53 to the immediately preceding claim, wherein the pre-tissue composition comprises human tissue cells.

62. The system of any one of claims 53 to the immediately preceding claim, wherein the pre-tissue composition comprises bovine, porcine, or poultry tissue cells.
